(19) United States
(12) Patent Application Publication
(10) Pub. No.: US 2010/0331650 A1
(43) Pub. Date: Dec. 30, 2010

(54) EPISODIC BLOOD GLUCOSE MONITORING SYSTEM WITH AN INTERACTIVE GRAPHICAL USER INTERFACE AND METHODS THEREOF

(75) Inventors: Carol J. Batman, Indianapolis, IN (US); Randy J. Gardner, Bloomington, IN (US); Amy S. Stanley, Noblesville, IN (US); Amy Day, Fishers, IN (US); Robert G. Davies, Carmel, IN (US); Mark Mears, Zionsville, IN (US); Stacia Davis, McCordsville, IN (US)

Correspondence Address: DINSMORE & SHOHL, LLP;FIFTH THIRD CENTER, ONE SOUTH MAIN STREET, SUITE 1300, DAYTON, OH, 45402, US

(73) Assignee: ROCHE DIAGNOSTICS OPERATIONS, INC., Indianapolis, IN (US)

(21) Appl. No.: 12/491,523

(22) Filed: Jun. 25, 2009

(51) Int. Cl. A61B 5/145

(52) U.S. Cl. 600/365

(57) ABSTRACT

A blood glucose monitoring system with a graphical user interface (GUI) and methods of controlling the system and performing episodic blood glucose testing using the GUI are disclosed. The system can comprise the GUI, an output display for displaying the GUI, user interfaces, a memory and a processor. An episodic blood glucose testing protocol can be programmed into the processor using the user interfaces and automatically saved into memory. A user can be alerted to test blood glucose levels based on the inputted testing protocol by the display of an alert icon on the GUI. Blood glucose level results can be received, displayed on the GUI along with a range icon and automatically saved into memory. Statistics of the saved blood glucose results can be calculated by the processor and saved into memory. The statistics can be retrieved for display on the GUI when prompted by the user.

### BACKGROUND

The present disclosure relates generally to a blood glucose monitoring system, and in particular, an episodic blood glucose monitoring system with an interactive graphical user interfaces (GUI) and methods thereof.

Diabetes is a disease typically associated with high levels of blood glucose resulting from defects in insulin production that causes sugar to build up in the body. According to the Centers of Disease Control, diabetes was the seventh leading cause of death in the United States in 2007 and can cause serious health complications including stroke and heart disease (e.g., diabetics have two to four time higher risk than those without diabetes), blindness (e.g., diabetes is the leading cause of blindness among adults 20-74 years old), kidney failure (e.g., diabetes is the leading cause) and lower-extremity amputations (e.g., 60% of nontraumatic amputations are performed on diabetics). As of June, 2008, nearly 24 million Americans (or nearly 8% of the population) were affected by diabetes. In 2007, the total direct and indirect cost of treating diabetes in the U.S. was approximately $174 billion.

Keeping blood glucose levels on target can prevent or delay the problems associated with diabetes. For example, general targets for diabetics can be to have blood glucose levels ranging between 80-140 mg/dL before meals and to have blood glucose levels of less than 180 mg/dL after meals. Studies in the United States and abroad have found improved glycemic control benefits people with either type 1 or type 2 diabetes. In general, every percentage point drop in A1c (the average amount of glucose in the blood during the past 2 to 3 months) blood test results can lower the risk of microvascular complications by 40%. Further, in people with type 1 diabetes, intensive insulin therapy has long-term beneficial effects in the risk of cardiovascular disease.

Typically, blood glucose levels can be checked using a blood glucose meter and a lancet. The lancet can be used to get a drop of blood from the diabetic patient which can be placed on a disposable strip. The strip is then inserted into the blood glucose meter to be read. The meter then can display the blood glucose level to the user, or patient, on a display screen. In general, the patient tests his/her blood glucose level before and after each meal and before bedtime. Patients also, typically, keeps a journal or log to keep track of their blood glucose levels. The date and time that the blood glucose level is measured is a typical entry into the journal. Notes are also made in the journal of things that may change the blood glucose levels, such as the size of the meal and energy level. Patient may graph their blood glucose levels to identify trends in blood glucose levels. The journal can be made available to a doctor or a health care team for analysis in order to recommend possible changes in diet, physical activity, or medicines to better control the patient's diabetes. However, in order for the journal to be beneficial, patients must remember to test blood glucose levels at the correct times as well as to accurately maintain the journal, in order for the patient and the patient's health care team to best control the patient's diabetes.

Therefore, there is a need for a blood glucose monitoring system with an interactive graphical user interface (GUI) that allows a patient to enter a blood glucose testing protocol, that alerts the patient of an upcoming blood glucose testing time, that allows the patient to enter testing conditions, that automatically maintains statistics and trends regarding the patient's blood glucose levels and that displays episodic blood glucose levels as well as blood glucose level statistics on the GUI in response to patient prompts.

### BRIEF SUMMARY

According to the present disclosure, a graphical user interface (GUI) for an episodic blood glucose monitoring system and methods of controlling the system and performing episodic blood glucose testing via the GUI are presented. The episodic blood glucose monitoring system can comprise the GUI, an output display for displaying the GUI and GUI icons associated with episodic blood glucose monitoring, user interfaces for accepting user input regarding testing protocol parameters, a memory for storing user input parameters and blood glucose testing results, and a processor that is in communication with the output device, the user interfaces and the memory. The processor can maintain the testing parameters inputted by the user and can calculate statistics related to the saved episodic blood glucose testing results. The GUI can display the GUI icons on the output display in accordance to the user inputted testing parameters and displays the statistical blood glucose testing results on the output display in response to a user prompt.

Accordingly, embodiments of the present disclosure may provide a blood glucose monitoring system with an interactive graphical user interface (GUI) that allows a patient to enter a blood glucose testing protocol, that automatically alerts the patient to an upcoming testing time, that allows the patient to enter testing conditions, that automatically maintains statistics and trends regarding the patient's blood glucose levels and that displays episodic blood glucose levels on the GUI in response to user prompts.

In one particular embodiment, an episodic blood glucose testing method is disclosed. The method comprises providing an episodic blood glucose monitoring system having a processor, memory, user interfaces, and an output display supporting a graphical user interface (GUI) which facilitates an episodic blood glucose testing mode; using the user interfaces and the GUI to enter the episodic blood glucose testing mode and to program the processor to execute an episodic blood glucose testing protocol comprised of a plurality of testing alert times; saving into memory the episodic blood glucose testing protocol; responding to a testing alert provided by the processor by inserting a test strip into the system, the testing alert corresponding to one of the testing alert times of the blood glucose testing protocol; conducting a blood glucose test using the inserted test strip; reviewing displayed blood glucose testing results provided along with a range indicator icon on the GUI; saving the blood glucose testing results into the memory; and prompting the processor to display calculated statistics of saved blood glucose testing results on the GUI.

In another embodiment, an episodic blood glucose monitoring system which uses a disposable test strip to determine a blood glucose level of a blood sample is disclosed. The system comprises a test strip reader; an output display which displays a graphical user interface (GUI) that has icons associated with an episodic blood glucose testing mode; user interfaces for accepting user input regarding the episodic blood glucose testing mode and an episodic blood glucose testing protocol comprised of a plurality of testing alert times for user specific episodic blood glucose testing; a memory which stores the episodic blood glucose testing protocol and any blood glucose testing results; a processor in communication with the test strip reader, output display, the user interfaces and the memory; and a computer program contained in memory having instructions which cause the processor to prompt on the output display for user input regarding setting times of each of the plurality of testing alert times, to display the icons associated with the episodic blood glucose testing mode on the output display in accordance to the user input accepted by the user interfaces, to provide a testing alert corresponding to one of the testing alert times of the blood glucose testing protocol, to conduct a blood glucose test upon insertion of the disposable test strip into the test strip reader, to display on the output display blood glucose testing results along with a range indicator icon, to save the blood glucose testing results into the memory, and to display on the output display calculated statistics of saved blood glucose testing results in response to additional user input accepted by the user interfaces.

In another embodiment, a method for controlling an episodic blood glucose monitoring system is disclosed. The method comprises allowing a user to toggle on the episodic blood glucose monitoring system between an episodic blood glucose testing mode in which an episodic blood glucose testing protocol, which prompts blood glucose testing and recording of meal information at user specified times, is automatically followed by the episodic blood glucose monitoring system and a normal blood glucose meter operations mode in which only a blood glucose testing results is displayed on an output display of the episodic blood glucose monitoring system after conducting a blood glucose test using a disposable test strip with the episodic blood glucose monitoring system; and displaying a graphical user interface that has icons associated with the episodic blood glucose testing mode on the output display of the episodic blood glucose monitoring system, wherein the icons comprises a pre-meal testing icon, a post-meal testing icon, and pre-bedtime testing icon.

In still another embodiment, an episodic blood glucose testing method is disclosed. The method comprises providing an episodic blood glucose monitoring system having a processor, memory, user interfaces, and an output display supporting a graphical user interface (GUI) which facilitates an episodic blood glucose testing mode; programming an episodic blood glucose testing protocol into the processor utilizing the user interfaces if the episodic blood glucose monitoring system is in the episodic testing mode; automatically saving the episodic blood glucose testing protocol into memory; alerting the user to test pre-meal blood glucose based on the inputted testing protocol by displaying a pre-meal alert icon on the GUI; inserting a blood glucose test strip containing a blood sample from a patient into the episodic blood glucose monitoring system; measuring a blood glucose level of the blood sample; displaying the blood glucose level measurement along with a blood glucose range indicator icon on the GUI; automatically saving the blood glucose level measurement into the memory; alerting the user to test post-meal blood glucose based on the inputted testing protocol by displaying a post-meal alert icon on the GUI after an elapse of a predetermined time after the pre-meal blood glucose measurement is saved; inserting a blood glucose test strip containing a blood sample from a patient into the episodic blood glucose monitoring system; measuring a blood glucose level of the blood sample; displaying the blood glucose level measurement along with a blood glucose range indicator icon on the GUI; automatically saving the blood glucose level measurement into the memory; inputting meal size after displaying blood glucose level measurement; automatically saving the meal size into the memory; alerting the user to test pre-bedtime blood glucose based on the inputted testing protocol by displaying a pre-bedtime alert icon on the GUI; inserting a blood glucose test strip containing a blood sample from a patient into the episodic blood glucose monitoring system; measuring a blood glucose level of the blood sample; displaying the blood glucose level measurement along with a blood glucose range indicator icon on the GUI; automatically saving the blood glucose level measurement into the memory; calculating statistics of saved blood glucose level measurements; retrieving statistics for display on the GUI when prompted by the user; and scrolling through the saved blood glucose measurements and calculated statistics using the user interfaces.

In another embodiment, an episodic blood glucose monitoring system with a graphical user interface (GUI) is disclosed. The system comprising: a power button to power the episodic blood glucose monitoring system on and off; a testing mode button to select for an episodic testing mode of the episodic blood glucose monitoring system; an output display for displaying the GUI; a left arrow button and a right arrow button to program an episodic blood glucose testing protocol into a processor if the episodic blood glucose monitoring system is in episodic testing mode; an alert icon displayed on the GUI to alert the user to test blood glucose based on the inputted testing protocol; a blood glucose test strip containing a blood sample from a patient to be inserted into the episodic blood glucose monitoring system for measuring a blood glucose level of the blood sample; a blood glucose range indicator icon to be displayed on the GUI along with a blood glucose level measurement; a meal size icon to prompt for meal size to be displayed on the GUI after displaying blood glucose level measurement for a post-meal testing; a memory to automatically save the episodic blood glucose testing protocol, blood glucose level measurements and meal size; and a processor to calculate statistics of saved blood glucose level measurements and to run other algorithms associated with the episodic blood glucose monitoring system.

In one specific embodiment, a graphical user interface (GUI) for an episodic blood glucose monitoring system is disclosed. The GUI comprises an episodic blood glucose testing mode icon associated with an episodic blood glucose testing mode of the episodic blood glucose monitoring system; an alert icon associated with a user specified testing time provided in an episodic blood glucose testing protocol followed by the episodic blood glucose monitoring system in the episodic blood glucose testing mode; an testing icon associated with one of a pre-meal testing, post-meal testing, and pre-bedtime testing; and an range indication icon associated with one of an acceptable blood glucose level range, a below acceptable blood glucose level range, and an above acceptable blood glucose level range.

Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a front view of an episodic blood glucose monitoring system according to an embodiment of the present invention;

FIG. 2 illustrates a side perspective view, in section, showing an interior of the episodic blood glucose monitoring system of FIG. 1;

FIG. 3 illustrates icons, symbols and indicia provided by a graphic user interface (GUI) of the episodic blood glucose monitoring system shown by FIG. 1 according to an embodiment of the present invention;

FIGS. 4A-F illustrate programming an episodic blood glucose testing protocol via the GUI of the episodic blood glucose monitoring system shown by FIG. 3 according to an embodiment of the present invention;

FIGS. 5A-D illustrate a pre-meal testing scenario supported by the GUI of the episodic blood glucose monitoring system shown by FIG. 3 according to an embodiment of the present invention;

FIGS. 6A-F illustrate a post-meal testing scenario supported by the GUI of the episodic blood glucose monitoring system shown by FIG. 3 according to an embodiment of the present invention;

FIGS. 7A-C illustrate the use of a snooze function supported by the GUI of the episodic blood glucose monitoring system shown by FIG. 3 according to an embodiment of the present invention; and

FIGS. 8A-N illustrate the use of memory/statistic review functions supported by the GUI of the episodic blood glucose monitoring system shown by FIG. 3 according to an embodiment of the present invention.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

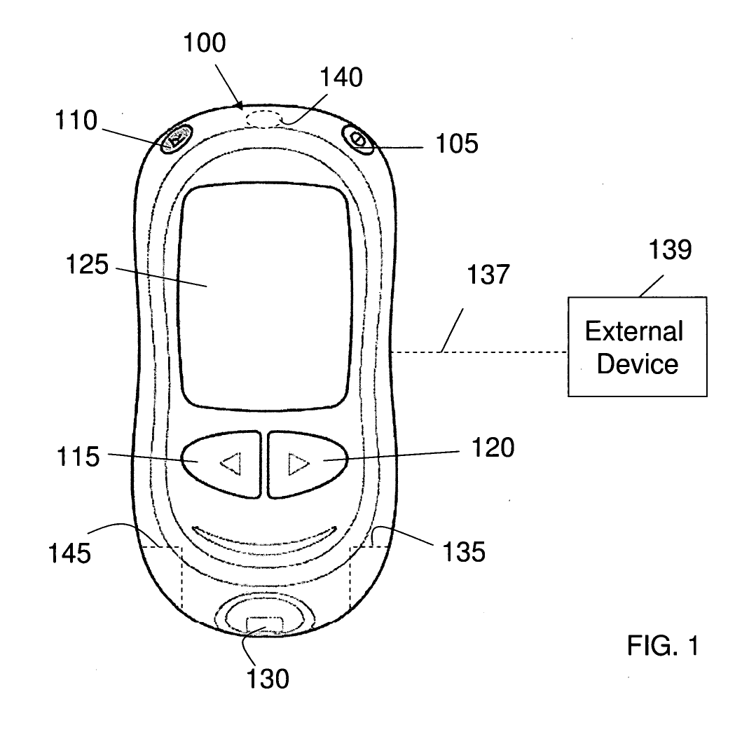

Referring initially to FIG. 1, one exemplary embodiment of an episodic blood glucose monitoring system 100 is illustrated. In this embodiment, as viewed exteriorly, the episodic blood glucose monitoring system 100 comprises hand-operated user interfaces 105, 110, 115, 120, an output display 125, and a strip port 130. In another embodiment, additionally a serial communications port 135 may provided in a side of the system 100 for communicating (i.e., transmit and receive data) 137 with an external device 139, such as a portable computer, a personal computer, a personal digital assistance, a cell phone, and the likes. In still another embodiment, optionally, a wireless interface device 140, such as based on IrDa or Bluetooth, may be provided to communicate with the external device if capable of such transmissions. Such a communication mode may be enter in one embodiment via a long press (i.e., greater then three seconds) on two of the user-interfaces, such as for example, user interfaces 115, 120.

A ROM key port 145 may be provided in an embodiment of the episodic blood glucose monitoring system 100 where calibration data for a lot of disposable test strips used with the system is provided on a ROM chip. It is to be appreciated in other embodiments, calibration data may be keyed into the system 100 via some combination of the user interfaces 105, 110, 115, 120, or received from an external device via either the port 135 or device 140.

In one exemplary embodiment, the user interfaces 105, 110, 115, 120 can comprise a power button 105, an episodic testing mode button 110, a left arrow button 115, and a right arrow button 120. The power button 105 when pressed can power the episodic blood glucose monitoring system 100 on or off. The episodic testing mode button 110 can be pressed by a user to toggle the episodic blood glucose monitoring system 100 between a normal blood glucose meter operations mode and an episodic blood glucose testing mode. It is to be appreciated that the system 100 in the normal blood glucose meter operations mode, functions to read a disposable test strip inserted into the strip port 130, to calculate the blood glucose level of a drop of blood placed on the test strip, and to display the level in mg/dl or mmol/l of the output display 125, as in a manner to mimic conventional blood glucose meters. Such a mode is useful in situations when a quick blood glucose reading is desired or when episodic blood glucose testing is not presently desired. Programming facilitating and scenarios illustrating the episodic blood glucose testing provided by system 100 is discussed hereafter in later sections. It is also to be appreciated that the method used to determine a patient's blood glucose level may be any known in the art such as, for example, measuring the amount of electricity that passes through the blood sample or measuring the amount of light reflects from the blood sample after the blood glucose reacts with the enzymes resident on the test strip. As such measurement methods are known to those skilled in the art, no further discussion regarding the same is provided herein.

By episodic blood glucose testing it is meant that an episodic blood glucose testing protocol, which prompts blood glucose testing and recording of meal information at user specified times, is followed automatically by the episodic blood glucose monitoring system 100.

Regarding the left and right arrow buttons 115,120, either or both can be pressed by the user to navigate various screens of the output display 125 and to select display features and/or functions. Although the term “button” is used when discussing the user interfaces 115, 120 which allow the user to manipulate the system 100, any suitable type of input device known in the art can be used such as, for example, and not to be limited thereto, sliders, knobs, switches, styli, trackballs, trackpoints, touch pads, and/or similar touchscreen features provided by the output display 125. By touchscreen, it is meant that the display 125 can detect the presence and location of a touch (finger or stylus) within the display area.

The output display 125 can be, for example, a display screen for displaying episodic blood glucose testing results and related graphical user interface (GUI) icons, symbols and/or indicia. The display screen 125 can be, for example, a liquid crystal display (LCD), an organic light emitting diode (OLED) display, a light emitting polymer (LEP) display, an organic electro luminescence (OEL) display, an electronic paper (E-paper) display, an interferometric modulator display (IMOD), or any suitable type of display known in the art. As mentioned above, the output display 125 may in one embodiment by a touchscreen which detects the presence and location of a touch (finger or stylus) within the display area. In such an embodiment, the left and right arrow buttons 115, 120 may not be provided as the output display 125 as a touchscreen would provide similar functionality. The various screens and displayable features i.e., GUI icons, symbols and/or indicia and functions of the output display 125 are discussed in greater details in later sections.

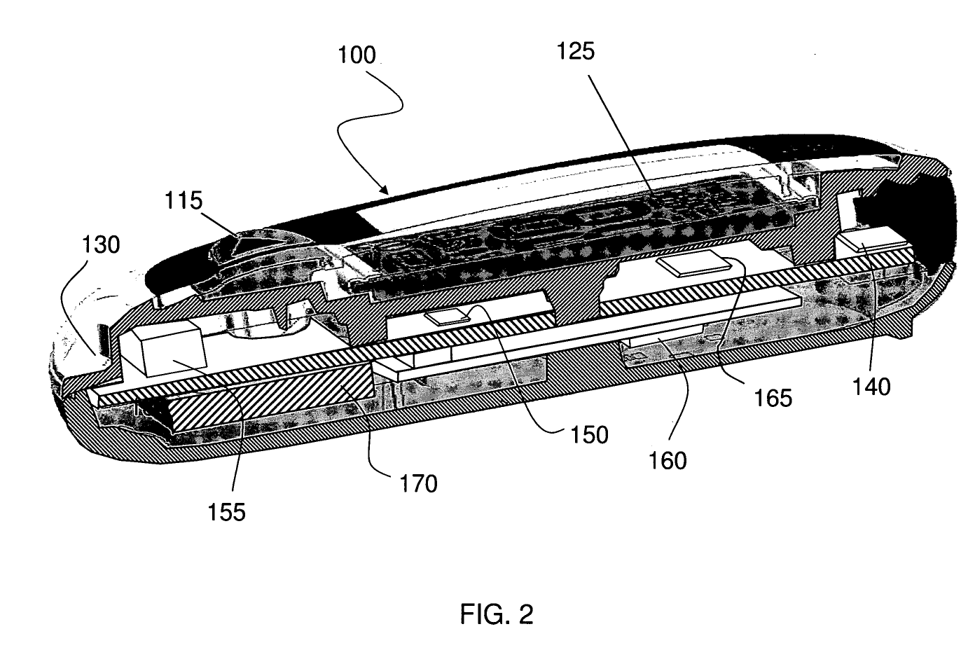

Turning now to FIG. 2, a side perspective view, in section, of the system 100 showing the interior features thereof is provided. As shown in the interior, the system 100 includes discrete circuit components, such as, memory 150, a strip reader 155 for reading a testing strip inserted into the strip port 130, and a serial communications controller 160, which are all electrically connected to a processor 165. A battery 170 is included to power the system 100. The memory 150 can be any suitable type of memory known in the art to store data on an appropriate computer readable medium, such as, for example, random-access memory (RAM), dynamic RAM, static RAM, cache, flash memory, memory sticks, virtual memory, or basic input/output system. The processor 165 can also be any suitable type of processor or ASIC known in the art capable of being programmed to implement the herein disclosed features of the blood glucose monitoring system 100. One such suitable blood glucose measuring system is disclosed by co-pending, a commonly owned, U.S. patent application Ser. No. 12/365,978, filed Feb. 5, 2009, entitled PORTABLE HANDHELD MEDICAL DIAGNOSTIC DEVICE HAVING A MEZZANINE CIRCUIT BOARD WITH A UNIVERSAL CONNECTION INTERFACE, and which the full disclosure is herein incorporated by reference.

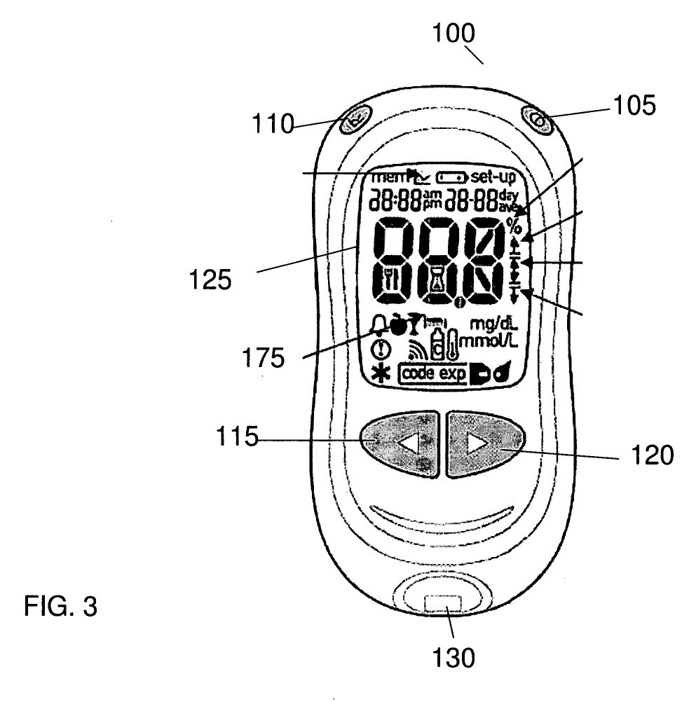

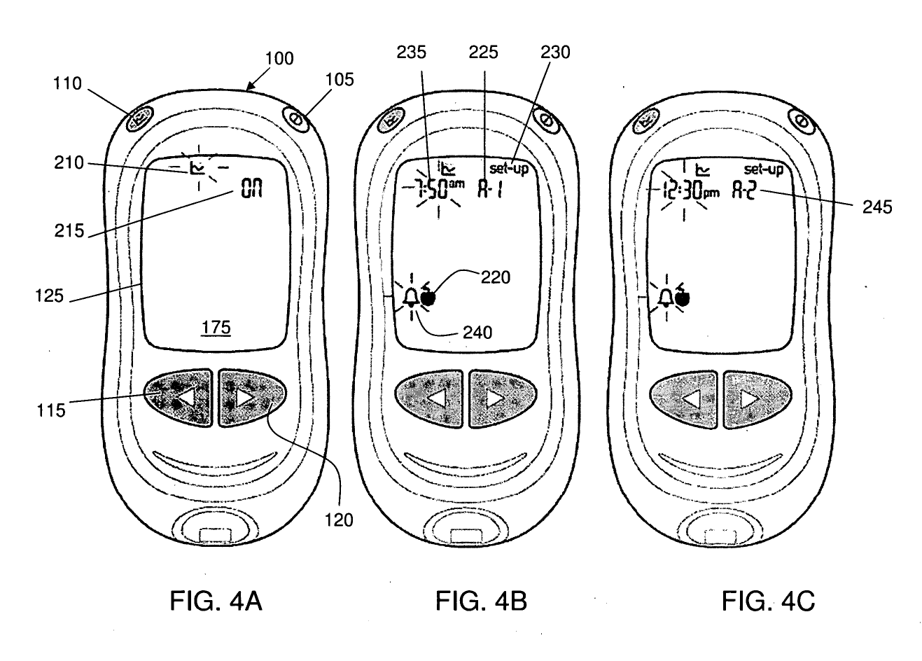

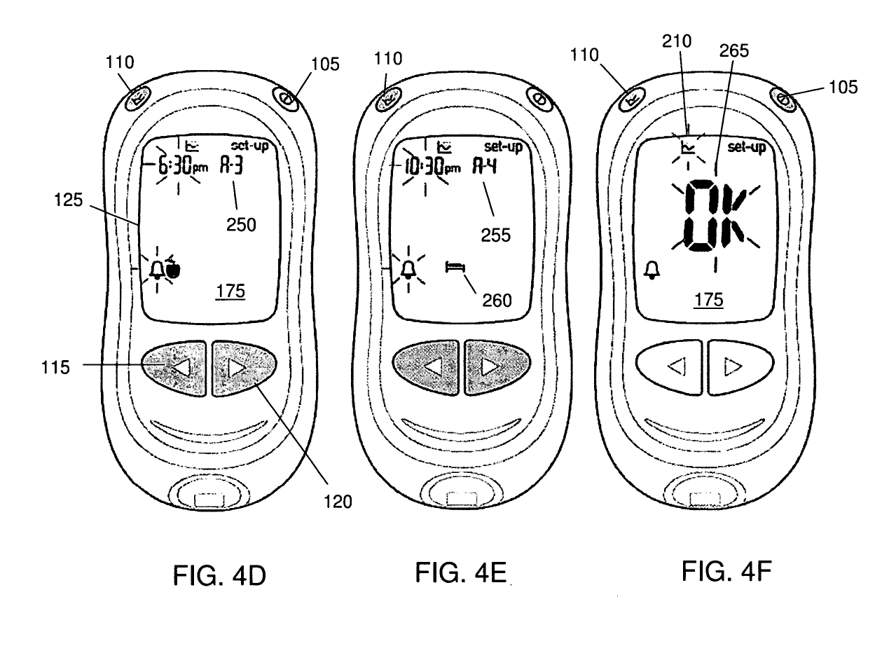

Turning to FIG. 3, an exemplary embodiment of a graphical user interface (GUI) 175 that can be utilized on the output display 125 by the episodic blood glucose monitoring system 100 is illustrated. The GUI 175 can be used to display various episodic testing icons and symbols as well as indicia such as, for example, blood glucose test results on the output display 125. The user, or patient, can set-up an individualized episodic protocol using the GUI 175 that can be programmed into the processor 165 and stored into memory 150 of the episodic blood glucose monitoring system 100. The programming of the episodic blood glucose testing protocol on the GUI 175 for the episodic blood glucose monitoring system 100 is illustrated by FIGS. 4A-F, which is now discussed hereafter.

In one exemplary embodiment, the user can begin the episodic testing protocol set-up by long pressing the testing mode button 110 in FIG. 4A. Long pressing can refer to pressing the testing mode button 110 for greater than a preset amount of time. In one exemplary embodiment, the preset amount of time can be three seconds or more. An episodic testing icon 210 can appear on the output display 125 along with “ON/OFF” indicia 215 as part of the GUI 175, which as shown is “ON” to indicate to the user that the episodic testing mode of the episodic blood glucose monitoring system 100 has now been entered. In one embodiment, the left and right arrow buttons 115, 120 can be pressed to toggle between “ON” or “OFF” indicia 215. If “OFF” is shown on the GUI 175 and the testing mode button 110 is pressed again, then the episodic testing protocol mode is exited and the system 100 functions as a conventional blood glucose measuring device or glucometer. However, if “ON” is shown on the GUI 175 and the testing mode button 110 is pressed, the set-up protocol then advances to setting a pre-breakfast alert 220 as seen in FIG. 4B. In another embodiment, a suitable combination of at least two of the user interfaces 105, 110, 115, 120 may also be used to exit the episodic testing protocol mode.

As shown by FIG. 4B, an apple icon 220 will be displayed along with an A-1 icon 225 and a set-up icon 230. The apple icon 220 is used to represent the pre-meal setting and the A-1 icon 225 is used to represent the pre-breakfast alert setting. An alert time 235 can be displayed as well as with an alert icon 240. In one exemplary embodiment, the alert time 235 and the alert icon 240 can flash to let the user know he/she is setting the pre-breakfast alert time. The alert time 235 can be changed by pressing the left and right arrow buttons 115, 120 to move the hour setting of the alert time 235 for the pre-breakfast setting to the desired hour. Once the desired hour is reached, the power button 105 can be pressed to move to the minute setting and save the hour setting into memory. The left and right arrow buttons 115, 120 can then be pressed to move the minute setting of the alert time 235 to the desired minute for the pre-breakfast alert. Once the desired minute is reached, the power button 105 can be pressed to move to the am/pm setting and save the minute setting into memory. The left and right arrow buttons 115, 120 can now be pressed to toggle between am and pm. Once the desired am/pm setting is reached, the power button 105 can be pressed and the am/pm setting is saved into memory. It is to be appreciated that error checking may be provided by the system 100 in one embodiment to ensure that a valid date-time has been entered for all of the herein disclosed alert times and dates.

Although not shown, in one embodiment, a series of alert dates (day/moth/year) can be also set by pressing the left and right arrow buttons 115, 120 to indicate a number of days over which the pre-breakfast alert will alarm. In such an embodiment, a default setting may be provided in which the pre-breakfast alert is provided daily unless a series has been entered. For example, in one embodiment, once a desired start date (day, month, year) is entered, the power button 105 can be pressed to save the start date into memory. The left and right arrow buttons 115, 120 can then be pressed to move the date to set a desired end date for the pre-breakfast alert. Once the desired date is reached, the power button 105 can be pressed to save the end date into memory. A similar testing series feature may also be provided to the other alert settings of the system 100 discussed hereafter in one embodiment, or in another embodiment may be a global setting for all alerts.

After setting up the pre-breakfast alert, pressing the testing mode button 110 advances the GUI 175 from the pre-breakfast setting, i.e. the A-1 icon 225, to a pre-lunch setting, which is represented by a A-2 icon 245 shown in FIG. 4C. As in the previous set-up for the pre-breakfast alert, the set-up icon 230 and the alert time 235 for the pre-lunch alert are also displayed as well as the apple icon 220 to indicate a pre-meal and the alert icon 240. As the set-up procedure for setting the alert time 235 for the pre-lunch alert is the same as the set-up produce for setting the pre-breakfast setting, for brevity, no further discussion is provided.

After the alert time 235 for the pre-lunch alert has been set and saved in memory, pressing the testing mode button 110 advances the GUI 175 to a pre-dinner setting, represented by the A-3 icon 250, as shown in FIG. 4D. As with the pre-lunch alert, the pre-dinner alert time can be set by using the left and right arrow buttons 115, 120 and power button 105 in the same manner as described above for the pre-breakfast alert time. Pressing the testing mode button 110 can again advance the GUI 175 to a pre-bedtime setting, represented by the A-4 icon 255, as is shown in FIG. 4E. In this exemplary embodiment, a bed icon 260 is displayed instead of the apple icon 220 as a visual reminder that the alert is for a pre-bedtime period instead of a pre-meal period. As with the other alerts, the pre-bedtime alert time can be set by using the left and right arrow buttons 115, 120 and power button 105 in the same manner as described above for the pre-breakfast alert time.

Pressing the testing mode button 110 one more time advances the GUI to an “OK” icon 265 shown in FIG. 4F. In one exemplary embodiment, the “OK” icon 265 and the episodic testing icon 210 can flash alternatively to visual indicate that all the alerts for the episodic protocol have been set. Pressing the power button 105 will exit the episodic testing programming mode of the GUI 175. Alternatively, the episodic blood glucose monitoring system 100 can time out after a pre-set time, such as, for example, a time selected from five to twenty seconds. In one exemplary embodiment, the alerts for the episodic protocol can be saved into memory after the “OK” icon 265 screen is reached. In one exemplary embodiment, the user may exit the programming of the episodic protocol alerts at any time by long pressing the power button 105. However, the episodic protocol alerts may not be saved into memory if the protocol alert programming is exited before the “OK” icon 265 is displayed.

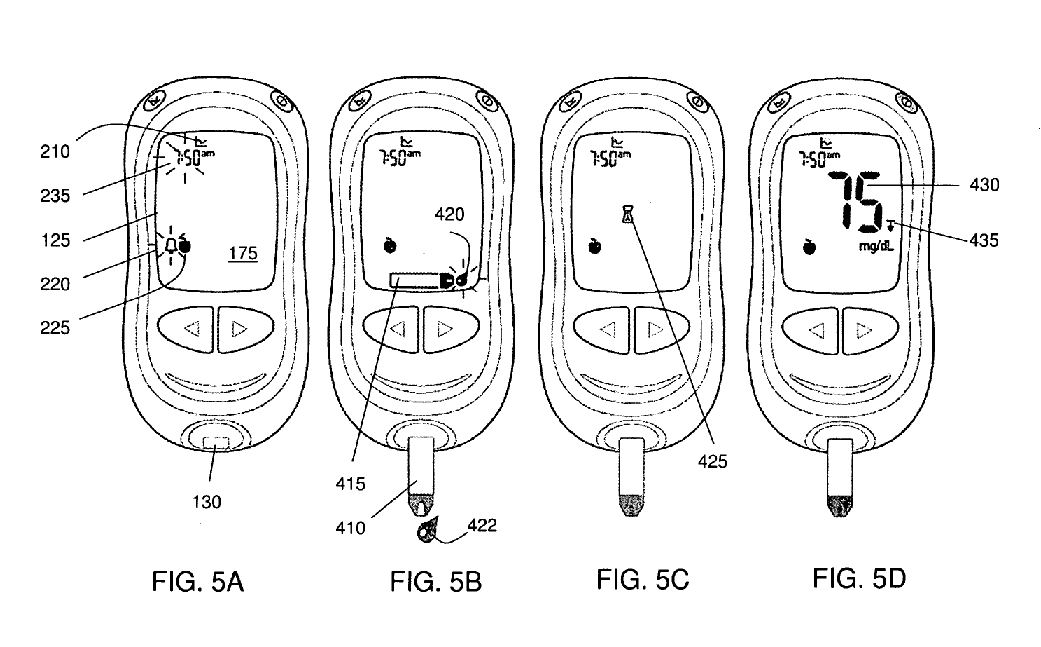

FIGS. 5A-D illustrate a typical pre-breakfast testing alert scenario. In FIG. 5A, at the pre-programmed pre-breakfast alert time 235, the output display 125 can display the alert icon 220 along with apple icon 225 as well as the episodic testing icon 210 to indicate a pre-meal testing time. In one exemplary embodiment, the alert can be visual, auditory, or both. In FIG. 5B, after the user inserts a test strip 410 into the strip port 130, the GUI 175 displays a strip icon 415 along with a blood drop icon 420 prompting the user to apply a blood sample 422 to the test strip 410 in FIG. 5B. The GUI 175 can display, in FIG. 5C, an hourglass icon 425 indicating that the processor 155 of the episodic blood glucose monitoring system 100 is measuring the blood glucose level of the patient's blood sample 422.

Once the blood glucose level of the blood sample 422 has been determined and saved into memory 150, the blood glucose level 430 can be displayed on the GUI 175 along with a range indicator icon 435 as illustrated in FIG. 5D. The range indicator icon 435 can graphically represent to the user whether the blood glucose level 430 of the blood sample 422 is within an acceptable range, below an acceptable range or above an acceptable range. In this exemplary embodiment, shown in FIG. 5D, the range indicator icon 435 indicates that the blood glucose level 430 of the blood sample 422 is below an acceptable range.

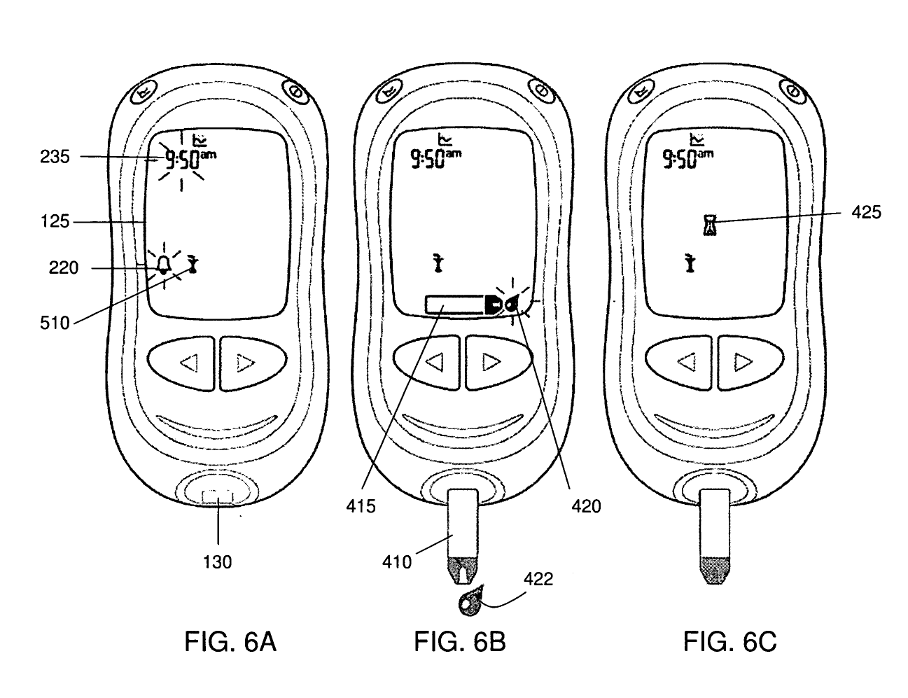

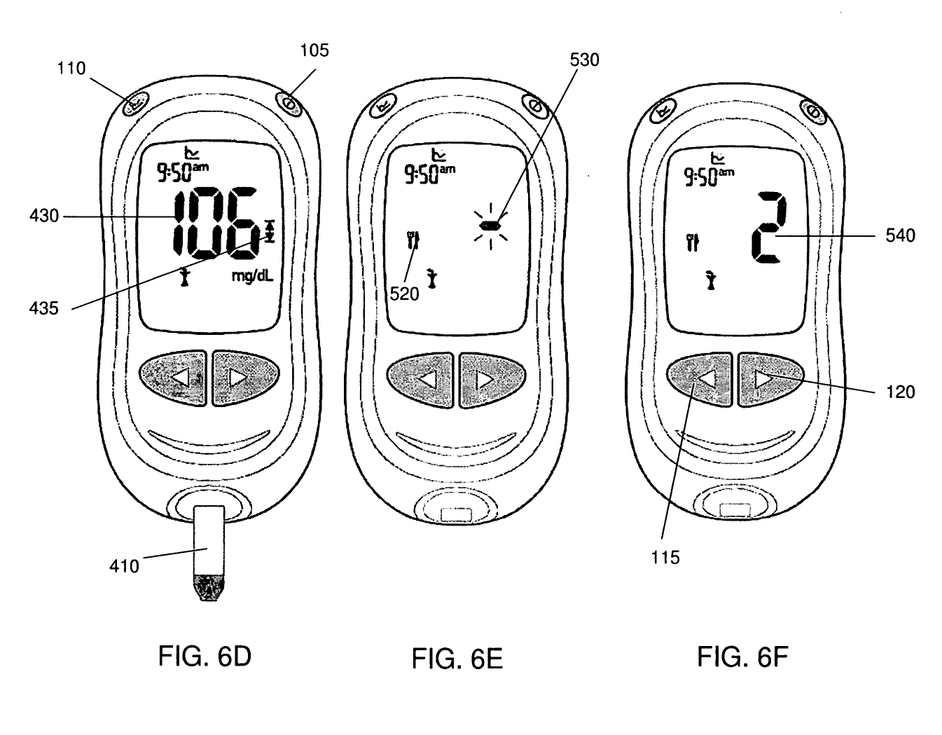

At a pre-determined time after the programmed pre-meal time alert, the user can be prompted to test a post-meal glucose level. In one exemplary embodiment, the pre-determined time can be a time selected from the range of about 90 to about 200 minutes, and in another embodiment may be set to 120 minutes. One possible post-meal alert scenario is illustrated in FIGS. 6A-F.

In FIG. 6A, at the pre-determined time, the output display 125 displays the alert icon 220 along with a post-meal icon 510 as well as an alert time 235 to indicate a post-meal testing time. In one exemplary embodiment, the post-meal icon 510 can be represented by an apple core, but in other embodiments may any other icon by which to indicate a post meal period. In one exemplary embodiment, the alert icon 220 and the post-meal alert time 235 can flash. In another exemplary embodiment, the episodic blood glucose monitoring system 100 may also provide an auditory alert.

After the alert, the user inserts a test strip 410 into the strip port 130. The GUI 175 then displays the strip icon 415 along with the blood drop icon 420 prompting the user to apply a blood sample 422 to the test strip 410 in FIG. 6B. The GUI 175 can display, in FIG. 6C, the hourglass icon 425 indicating that the processor 155 of the blood glucose monitoring system 100 is measuring the blood glucose level of the blood sample. Once the blood glucose level of the blood sample 422 has been determined and saved into memory 150, the blood glucose level 430 can be displayed on the GUI 175 along with the range indicator icon 435 as shown in FIG. 6D. In this exemplary example, the range indicator icon 435 indicates that the blood glucose level 430 of the blood sample 422 is within an acceptable range.

After user removes the test strip 410 or presses any one of the user interfaces, a meal icon 520 can appear along with a user prompt 530 to enter a meal size as shown in FIG. 6E. In one embodiment, meal sizes small, medium, and large is represented by the number 1, 2, and 3, respectively. In other embodiment, other indicia, symbols or icons may be used to identify such meal sizes. The user can use the left and right arrow buttons 115, 120 to enter the meal size 540, which is indicated in this example, as being a medium “2” meal size as shown in FIG. 6F. The meal size 540 is then saved into memory 150. Although not shown for brevity, information concerning meal speed may also be enter after the meal speed in a similar manner, where slow, medium, and fast meal speeds may also be represented by the numbers 1, 2, and 3, respectively. The user can then remove the test strip 410, if not previously done so, and press the power button 105 or can wait until the episodic blood glucose monitoring system 100 times out in order to exit the post-meal testing scenario. In one exemplary embodiment, the episodic blood glucose monitoring system 100 can time out in about five seconds.

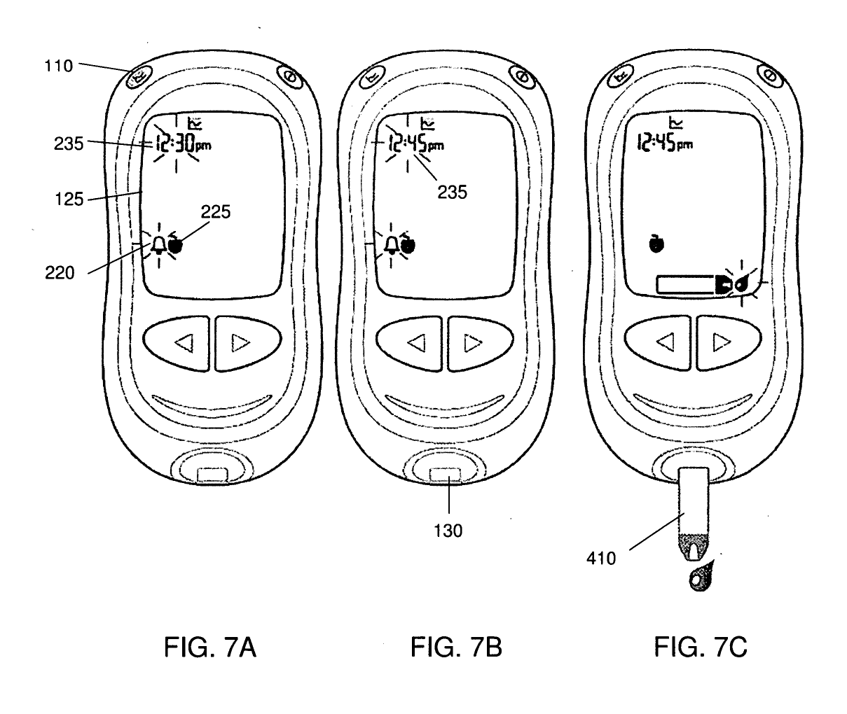

FIGS. 7A-C illustrate an exemplary snooze feature of the episodic blood glucose monitoring system 100. In FIG. 7A, the alert icon 220 for a pre-lunch testing can be displayed on the GUI 175 at the pre-programmed pre-lunch time. If the user prefers not to test at this time, the testing mode button 110 can be pressed to delay testing by a preset amount of time. In one exemplary example, the preset amount of time can be a time selected from the range of 10 to 30 minutes, and in another embodiment the time is approximately 15 minutes. After the expiration of the preset amount of time, the user can again be alerted to test wherein the alert time 235 has advance now fifteen minutes as is shown in FIG. 7B. The user can continue to delay the testing by the preset amount of time increments until a preset maximum delay is reached where testing will be required by the user in order clear and/or silence the testing alert icon 220.

In one exemplary embodiment, the preset maximum delay can be approximately one hour. This delay of testing is referred to herein as a snooze function of the system 100. To turn off either the snooze function if in a delay period or the alert icon 220 and begin testing, the user can insert a test strip 410 to initiate the blood glucose testing as shown in FIG. 7C. As the testing and presentation of the test results are the same as previously discuss above in reference to FIGS. 5A-D, no further discussion is provided.

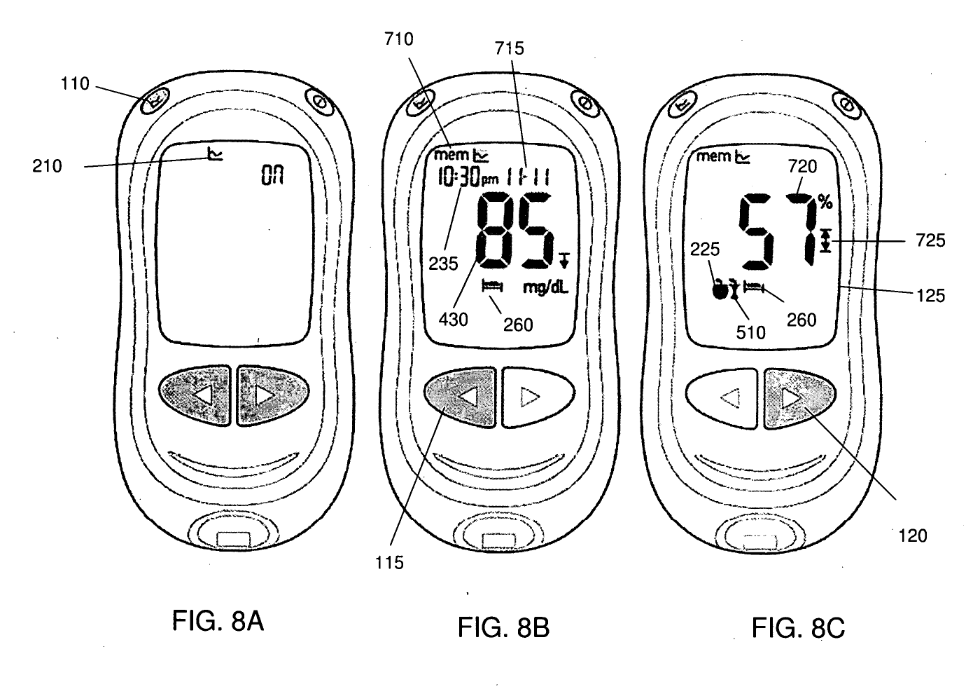

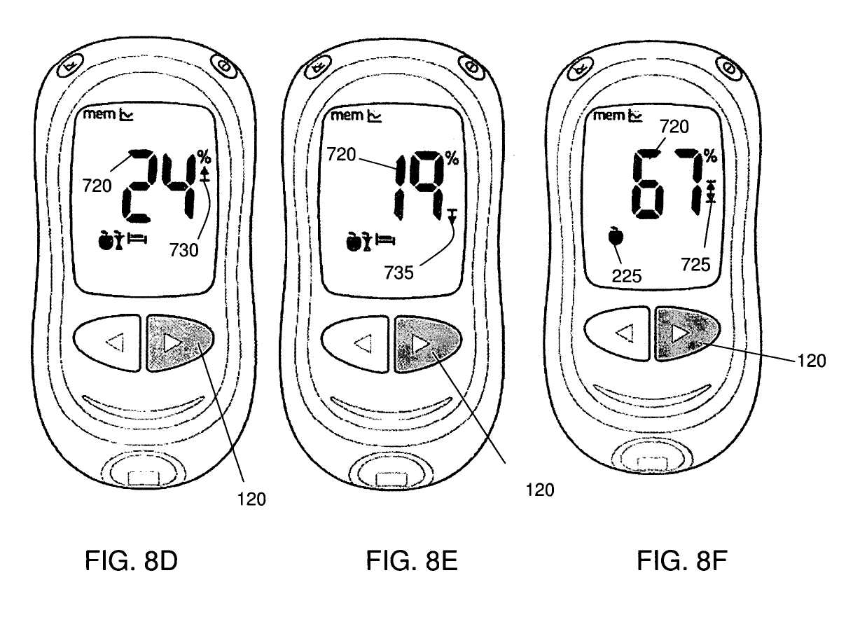

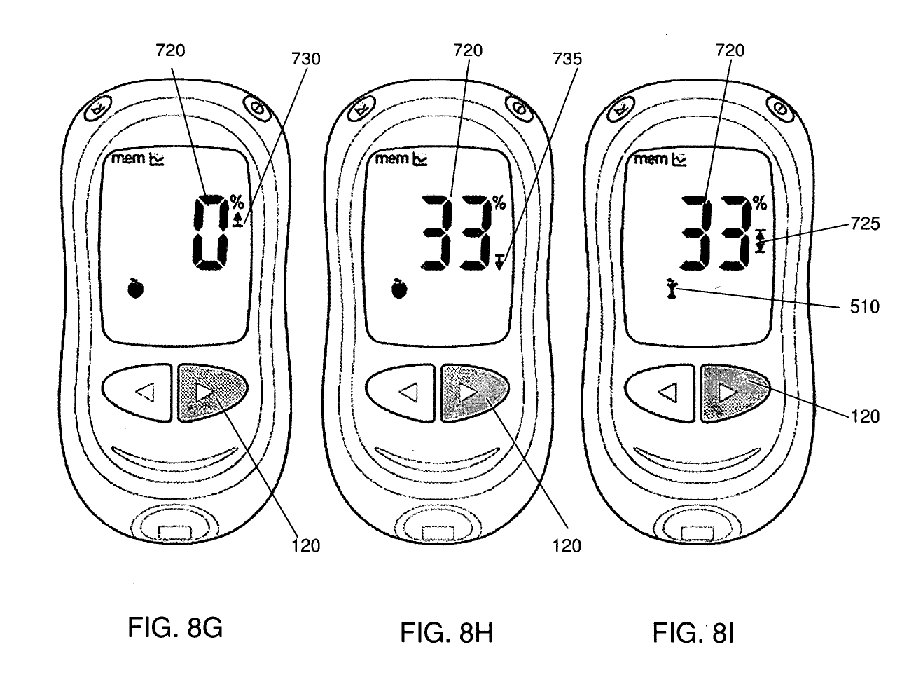

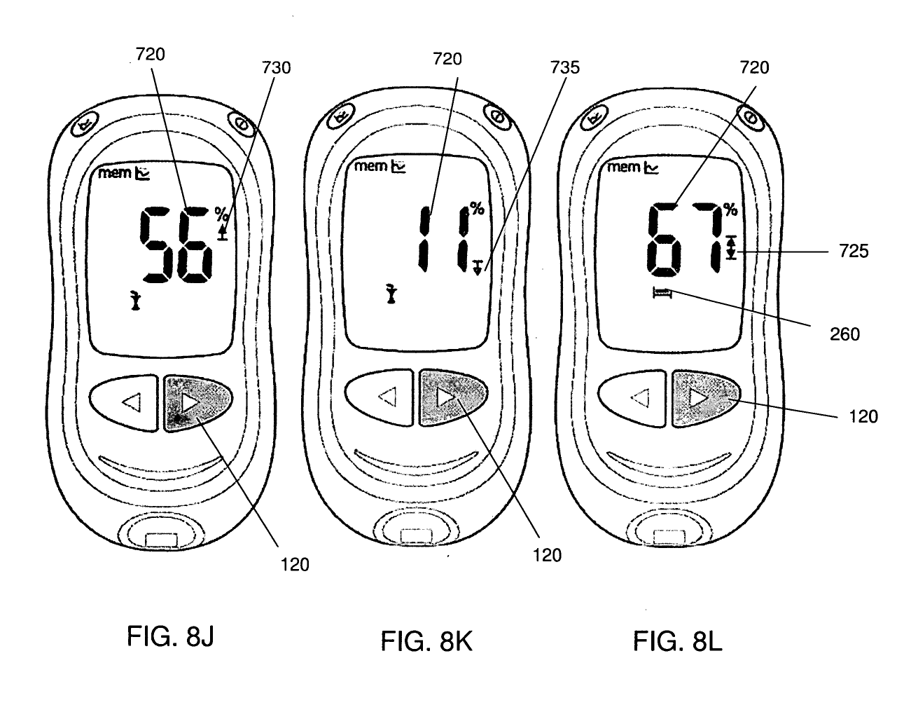

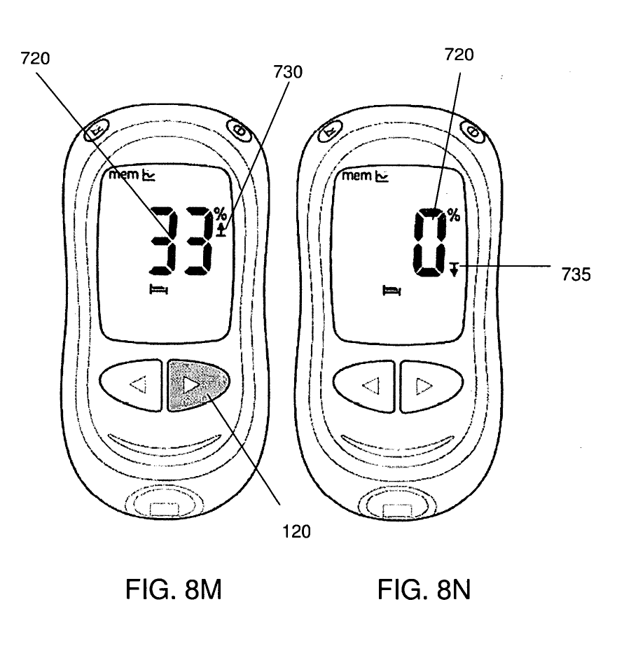

FIGS. 8A-N illustrate a memory/statistic review display of the episodic blood glucose testing results stored in the memory 150 of the episodic blood glucose monitoring system 100. The user can begin the statistical review of the stored blood glucose results by short pressing the testing mode button 110 as shown in FIG. 8A. Short pressing can refer to the pressing the testing mode button 110 for less than a preset amount of time. In one exemplary embodiment, the preset amount of time can be less than three seconds. In FIG. 8B, the memory icon 710 can appear next to episodic testing icon 210 along with a date 715 and time 716 of the individual test results and the type of test i.e., pre-meal, post-meal or pre-bedtime, via a displayed respective test icons 225, 510, 260. If the user presses the left arrow button 115, the user can scroll chronologically through the individual results of that particular episodic test protocol, which in the illustrated embodiment would be all pre-bedtime test result as indicated by the displayed pre-bedtime icon 260.

If the right arrow button 120 is pressed, the user can scroll forward through the episodic testing statistics calculated by the processor 155 from the blood glucose testing results stored in the memory 150. The first statistical GUI display shown in FIG. 8C can illustrate the percentage of the total number of blood glucose testing results that were within the target blood glucose range by displaying the percentage 720 along with a “within an accepted” range indicator icon 725 and all three test icons 225, 510, 260 (i.e., pre-meal, post-meal and pre-bedtime) on the output display 125. Pressing the right arrow button 120 can take the user to the next statistical screen illustrated in FIG. 8D which illustrates the percentage 720 of total tests that were above the target range by displaying an “above an accepted” range indicator icon 730. Pressing the right arrow button 120 next can take the user to the next statistical screen illustrated in FIG. 8E which illustrates the percentage 720 of total tests that were below the target range by displaying a “below an accepted” range indicator icon 735. To scroll backwards through the episodic testing statistics, the user can press the left arrow button 115.

After the screens showing the percentages of the total number of blood glucose testing results in each range, pressing the right arrow button 120 again can take the user to the next set of statistical screens for only the pre-meal tests which can be represented by displaying the apple icon 225. The first screen is illustrated in FIG. 8F and it can display the percentage 720 of pre-meal tests that were within target range by displaying the within an accepted range indicator icon 725. Pressing the right arrow button 120 again can take the user to the next statistical screen illustrated in FIG. 8G which shows the percentage 720 of pre-meal tests that were above range by displaying the above an accepted range indicator icon 730 and pressing the right arrow button 120 can take the user to the next statistical screen illustrated in FIG. 8H which displays the percentage 720 of pre-meal tests below range by displaying the below an accepted range indicator icon 735. Again, to scroll backwards through the episodic testing statistics, the user can press the left arrow button 115 to move to the previously displayed screens.

After the set of statistical screens for only the pre-meal tests, pressing the right arrow button 120 again can take the user to the next set of statistical screens for only the post-meal tests which can be represented by displaying the apple core, or post-meal icon 510. The first screen is illustrated in FIG. 8I and it can display the percentage 720 of post-meal tests that were within target range by displaying the within an accepted range indicator icon 725. Pressing the right arrow button 120 again can take the user to the next statistical screen illustrated in FIG. 8J which shows the percentage 720 of post-meal tests above range by displaying the above an accepted range indicator icon 730. Pressing the right arrow button 120 again can take the user to the next statistical screen illustrated in FIG. 8K which displays the percentage 720 of post-meal tests below range by displaying the below an accepted range indicator icon 735. Once again, to scroll backwards through the episodic testing statistics, the user can press the left arrow button 115 to move to the previously displayed screens.

Finally, after the set of statistical screens for only the post-meal tests, pressing the right arrow button 120 again can take the user to the next set of statistical screens for only the pre-bedtime test represented by the bed icon 260. The first screen is illustrated in FIG. 8L and it can displays the percentage 720 of pre-bedtime tests that were within target range by displaying the within an accepted range indicator icon 725. Pressing the right arrow button 120 can take the user to the next statistical screen illustrated in FIG. 8M which shows the percentage 720 of pre-bedtime tests above range by displaying the above an accepted range indicator icon 730 and pressing the right arrow button 120 can take the user to the next statistical screen illustrated in FIG. 8N which displays the percentage 720 of pre-bedtime tests below range by displaying the below an accepted range indicator icon 735. And again, to scroll backwards through the episodic testing statistics, the user can press the left arrow button 115 to move to the previously displayed screens. In other embodiments, similar statistical screens may be provided by the system 100 which display a 7-, 14-, 30-, and 90-day bG averages of all the blood glucose test results as well as a 7-, 14-, 30-, and 90-day bG averages for all the pre-meal and also all the post meal tests stored in memory 150.

It is to be appreciated that all the above disclosed processes and functions provided by the episodic blood glucose monitoring system 100 result from a computer program stored in memory, such as memory 150, that has instructions which when executed by the processor 165 create means for implementing all the processes and functions disclosed above in reference to FIGS. 3-8. For example, in one embodiment, the computer program has instructions that will cause the processor 165 to prompt on the output display 125 for user input regarding setting times of each of the plurality of testing alert times, to display the icons associated with the episodic blood glucose testing mode on the output display 125 in accordance to the user input accepted by the user interfaces 105, 110, 115, 120, to provide a testing alert corresponding to one of the testing alert times of the blood glucose testing protocol, to conduct a blood glucose test upon insertion of the disposable test strip into the test strip reader, to display on the output display 125 blood glucose testing results along with a range indicator icon, to save the blood glucose testing results into the memory, and to display on the output display 125 calculated statistics of saved blood glucose testing results in response to additional user input accepted by the user interfaces 105, 110, 115, 120.

In another embodiment, the computer program further comprises instructions which cause the processor 165 to toggle, upon further user input accepted by the user interfaces 110, 115, 120, between the episodic blood glucose testing mode and a normal blood glucose meter operations mode of the episodic blood glucose monitoring system in which the processor 165 only displays the blood glucose testing results on the output display 125 after conducting the blood glucose test on the inserted disposable test strip. In another embodiment, the computer program further comprises instructions which cause the processor 165 to reset the testing alert for a set period of time upon further user input accepted by the user interfaces 110, 115, 120 and to re-alert the testing alert after elapse of the set period of time. In still another embodiment, the computer program further comprises instructions which cause the processor 165 to prompt on the output display 125 user input which designates each of the testing alert times as a pre-meal testing, a post-meal testing, or a pre-bedtime testing.

In still another embodiment, the computer program further comprises instructions which cause the processor 165 to prompt on the output display 125 user input concerning at least one of a meal size and a meal speed for the blood glucose test if the alert time was for a post-meal test. In yet still another embodiment, the computer program further comprises instructions which cause the processor 165 to calculate for the calculated statistics what percentage of blood glucose testing results fall within an acceptable blood glucose level range, below an acceptable blood glucose level range, and above an acceptable blood glucose level range.

In another embodiment, the computer program further comprises instructions which cause the processor 165 to calculate for the calculated statistics what percentage of blood glucose testing results fall within an acceptable blood glucose level range, below an acceptable blood glucose level range, and above an acceptable blood glucose level range for pre-meal blood glucose testing, for post-meal blood glucose testing, and for pre-bedtime testing. The computer program for implementing the present invention may be written in various object-oriented programming languages, such as Delphi and Java. RTM. However, it is understood that other object oriented programming languages, such as C++ and Smalltalk, as well as conventional programming languages, such as FORTRAN or COBOL, could be utilized without departing from the spirit and intent of the present invention.

Having the testing protocol and the blood glucose test results automatically saved into memory 150 of the episodic blood glucose monitoring system 100 can help increase user convenience since the user no longer needs to maintain a separate testing schedule and/or journal. Further, automatically saving the results may help reduce the number of errors that may result from having to keep a journal separate from the episodic blood glucose monitoring system 100. The GUI 175 of the output display 125 can also provide the user with interpretative context for his or her own blood glucose data such as, for example, meal size, meal speed, and instant summaries of statistics regarding the blood glucose levels being within, above or under an acceptable range. Finally, having the contextualized data resident on the episodic blood glucose monitoring system 100 can help improve interaction with the user's doctor or health care professional to assist in improving therapy decisions, thereby leading to better patient outcomes.

Having described the disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

What is claimed is:

1. An episodic blood glucose testing method comprising:
providing an episodic blood glucose monitoring system having a processor, memory, user interfaces, and an output display supporting a graphical user interface (GUI) which facilitates an episodic blood glucose testing mode;
using the user interfaces and the GUI to enter the episodic blood glucose testing mode and to program the processor to execute an episodic blood glucose testing protocol comprised of a plurality of testing alert times;
saving into memory the episodic blood glucose testing protocol,
responding to a testing alert provided by the processor by inserting a test strip into the system, the testing alert corresponding to one of the testing alert times of the blood glucose testing protocol;
conducting a blood glucose test using the inserted test strip;
reviewing displayed blood glucose testing results provided along with a range indicator icon on the GUI;
saving the blood glucose testing results into the memory; and
prompting the processor to display calculated statistics of saved blood glucose testing results on the GUI.

2. The method of claim 1, further comprising toggling between the episodic blood glucose testing mode and a normal blood glucose meter operations mode of the episodic blood glucose monitoring system in which only the displaying of the blood glucose testing results is provided on the output display after conducting the blood glucose test on the inserted test strip.

3. The method of claim 1, further comprising the processor automatically saving into memory the episodic blood glucose testing protocol and the blood glucose results.

4. The method of claim 1, further comprising resetting the testing alert for a set period of time.

5. The method of claim 1, further comprising responding to a re-alerting of the testing alert after elapse of a set period of time.

6. The method of claim 1, further comprising using the user interfaces and GUI to program the episodic blood glucose testing protocol by setting a time for each of the testing alert times and designating each of the testing alert times as a pre-meal testing, a post-meal testing, or a pre-bedtime testing.

7. The method of claim 1, wherein the testing alert comprises an alert icon displayed on the output display along with an icon indicating one of a pre-meal testing, post-meal testing, and pre-bedtime testing.

8. The method of claim 1, further comprising using the user interfaces and the GUI to enter and save in memory at least one of a meal size and a meal speed for the blood glucose test if the alert time was for a post-meal test.

9. The method of claim 1, wherein the calculated statistics comprise the processor calculating what percentage of blood glucose testing results fall within an acceptable blood glucose level range, below an acceptable blood glucose level range, and above an acceptable blood glucose level range.

10. The method of claim 1, wherein the calculated statistics comprise the processor calculating what percentage of blood glucose testing results fall within an acceptable blood glucose level range, below an acceptable blood glucose level range, and above an acceptable blood glucose level range for pre-meal blood glucose testing, for post-meal blood glucose testing, and for pre-bedtime testing.

11. The method of claim 1, further comprising viewing the saved blood glucose testing results and calculated statistics.

12. The method of claim 11, wherein the saved blood glucose testing results and calculated statistics can be viewed by scrolling through the results and statistics on the output display.

13. The method of claim 1, further comprising providing data comprising the blood glucose testing results and the calculated statistics saved on the episodic blood glucose monitoring system to an external device.

14. An episodic blood glucose monitoring system which uses a disposable test strip to determine a blood glucose level of a blood sample, said system comprising:
a test strip reader;
an output display which displays a graphical user interface (GUI) that has icons associated with an episodic blood glucose testing mode;
user interfaces for accepting user input regarding the episodic blood glucose testing mode and an episodic blood glucose testing protocol comprised of a plurality of testing alert times for user specific episodic blood glucose testing;
a memory which stores the episodic blood glucose testing protocol and any blood glucose testing results;
a processor in communication with the test strip reader, output display, the user interfaces and the memory; and
a computer program contained in memory having instructions which cause the processor to prompt on the output display for user input regarding setting times of each of the plurality of testing alert times, to display the icons associated with the episodic blood glucose testing mode on the output display in accordance to the user input accepted by the user interfaces, to provide a testing alert corresponding to one of the testing alert times of the blood glucose testing protocol, to conduct a blood glucose test upon insertion of the disposable test strip into the test strip reader, to display on the output display blood glucose testing results along with a range indicator icon, to save the blood glucose testing results into the memory, and to display on the output display calculated statistics of saved blood glucose testing results in response to additional user input accepted by the user interfaces.

15. The episodic blood glucose monitoring system of claim 14, wherein the episodic blood glucose monitoring system is battery operated.

16. The episodic blood glucose monitoring system of claim 14, wherein the episodic blood glucose monitoring system is capable of communicating with an external device.

17. The episodic blood glucose monitoring system of claim 14, wherein the computer program further comprises instructions which cause the processor to toggle, upon further user input accepted by the user interfaces, between the episodic blood glucose testing mode and a normal blood glucose meter operations mode of the episodic blood glucose monitoring system in which the processor only displays the blood glucose testing results on the output display after conducting the blood glucose test on the inserted disposable test strip.

18. The episodic blood glucose monitoring system of claim 14, wherein the computer program further comprises instructions which cause the processor to reset the testing alert for a set period of time upon further user input accepted by the user interfaces and to re-alert the testing alert after elapse of the set period of time.

19. The episodic blood glucose monitoring system of claim 14, wherein the computer program further comprises instructions which cause the processor to prompt on the output display user input which designates each of the testing alert times as a pre-meal testing, a post-meal testing, or a pre-bedtime testing.

20. The episodic blood glucose monitoring system of claim 14, wherein the testing alert comprises an alert icon displayed on the output display along with an icon indicating one of a pre-meal testing, post-meal testing, and pre-bedtime testing.

21. The episodic blood glucose monitoring system of claim 14, wherein the computer program further comprises instructions which cause the processor to prompt on the output display user input concerning at least one of a meal size and a meal speed for the blood glucose test if the alert time was for a post-meal test.

22. The episodic blood glucose monitoring system of claim 14, wherein the computer program further comprises instructions which cause the processor to calculate for the calculated statistics what percentage of blood glucose testing results fall within an acceptable blood glucose level range, below an acceptable blood glucose level range, and above an acceptable blood glucose level range.

23. The episodic blood glucose monitoring system of claim 14, wherein the computer program further comprises instructions which cause the processor to calculate for the calculated statistics what percentage of blood glucose testing results fall within an acceptable blood glucose level range, below an acceptable blood glucose level range, and above an acceptable blood glucose level range for pre-meal blood glucose testing, for post-meal blood glucose testing, and for pre-bedtime testing.

24. The episodic blood glucose monitoring system of claim 14, wherein the icons comprise icons representing alerts, status, type of test, range indicator, user prompts, and the GUI further comprises indicia and symbols representing time, a date, and a blood glucose level.

25. The episodic blood glucose monitoring system of claim 14, wherein the user interfaces comprises a left arrow input device, a right arrow input device, a testing mode input device, and a power input device.

26. A method for controlling an episodic blood glucose monitoring system, the method comprising:
allowing a user to toggle on the episodic blood glucose monitoring system between an episodic blood glucose testing mode in which an episodic blood glucose testing protocol, which prompts blood glucose testing and recording of meal information at user specified times, is automatically followed by the episodic blood glucose monitoring system and a normal blood glucose meter operations mode in which only a blood glucose testing results is displayed on an output display of the episodic blood glucose monitoring system after conducting a blood glucose test using a disposable test strip with the episodic blood glucose monitoring system; and
displaying a graphical user interface that has icons associated with the episodic blood glucose testing mode on the output display of the episodic blood glucose monitoring system, wherein the icons comprises a pre-meal testing icon, a post-meal testing icon, and pre-bedtime testing icon.

27. The method according to claim 26, further comprising using a computer program which has instructions which cause the episodic blood glucose monitoring system to prompt on the output display for user input regarding setting times of a plurality of testing alert times which are used by the episodic blood glucose monitoring system in the episodic blood glucose testing mode to prompt the blood glucose testing and the recording of meal information at the user specified times, to display the icons associated with the episodic blood glucose testing mode on the output display, to provide a testing alert corresponding to one of the testing alert times of the blood glucose testing protocol, to conduct a blood glucose test upon insertion of the disposable test strip into the episodic blood glucose monitoring system, to display on the output display blood glucose testing results along with a range indicator icon, to save the blood glucose testing results into memory, and to display on the output display calculated statistics of saved blood glucose testing results in response to additional user input accepted by the episodic blood glucose monitoring system.

28. An episodic blood glucose testing method comprising:
providing an episodic blood glucose monitoring system having a processor, memory, user interfaces, and an output display supporting a graphical user interface (GUI) which facilitates an episodic blood glucose testing mode;
programming an episodic blood glucose testing protocol into the processor utilizing the user interfaces if the episodic blood glucose monitoring system is in the episodic testing mode;
automatically saving the episodic blood glucose testing protocol into memory;
alerting the user to test pre-meal blood glucose based on the inputted testing protocol by displaying a pre-meal alert icon on the GUI;
inserting a blood glucose test strip containing a blood sample from a patient into the episodic blood glucose monitoring system;
measuring a blood glucose level of the blood sample;
displaying the blood glucose level measurement along with a blood glucose range indicator icon on the GUI;
automatically saving the blood glucose level measurement into the memory;
alerting the user to test post-meal blood glucose based on the inputted testing protocol by displaying a post-meal alert icon on the GUI after an elapse of a predetermined time after the pre-meal blood glucose measurement is saved;
inserting a blood glucose test strip containing a blood sample from a patient into the episodic blood glucose monitoring system;
measuring a blood glucose level of the blood sample;
displaying the blood glucose level measurement along with a blood glucose range indicator icon on the GUI;
automatically saving the blood glucose level measurement into the memory;
inputting meal size after displaying blood glucose level measurement;
automatically saving the meal size into the memory;
alerting the user to test pre-bedtime blood glucose based on the inputted testing protocol by displaying a pre-bedtime alert icon on the GUI;
inserting a blood glucose test strip containing a blood sample from a patient into the episodic blood glucose monitoring system;
measuring a blood glucose level of the blood sample;
displaying the blood glucose level measurement along with a blood glucose range indicator icon on the GUI;
automatically saving the blood glucose level measurement into the memory;
calculating statistics of saved blood glucose level measurements;
retrieving statistics for display on the GUI when prompted by the user; and
scrolling through the saved blood glucose measurements and calculated statistics using the user interfaces.

29. An episodic blood glucose monitoring system with a graphical user interface (GUI), the system comprising:
a power button to power the episodic blood glucose monitoring system on and off;
a testing mode button to select for an episodic testing mode of the episodic blood glucose monitoring system;
an output display for displaying the GUI;
a left arrow button and a right arrow button to program an episodic blood glucose testing protocol into a processor if the episodic blood glucose monitoring system is in episodic testing mode;
an alert icon displayed on the GUI to alert the user to test blood glucose based on the inputted testing protocol;
a blood glucose test strip containing a blood sample from a patient to be inserted into the episodic blood glucose monitoring system for measuring a blood glucose level of the blood sample;
a blood glucose range indicator icon to be displayed on the GUI along with a blood glucose level measurement;
a meal size icon to prompt for meal size to be displayed on the GUI after displaying blood glucose level measurement for a post-meal testing;
a memory to automatically save the episodic blood glucose testing protocol, blood glucose level measurements and meal size; and
a processor to calculate statistics of saved blood glucose level measurements and to run other algorithms associated with the episodic blood glucose monitoring system.

30. A graphical user interface (GUI) for an episodic blood glucose monitoring system, the GUI comprising:
an episodic blood glucose testing mode icon associated with an episodic blood glucose testing mode of the episodic blood glucose monitoring system;
an alert icon associated with a user specified testing time provided in an episodic blood glucose testing protocol followed by the episodic blood glucose monitoring system in the episodic blood glucose testing mode;
an testing icon associated with one of a pre-meal testing, post-meal testing, and pre-bedtime testing; and
an range indication icon associated with one of an acceptable blood glucose level range, a below acceptable blood glucose level range, and an above acceptable blood glucose level range.
